(19) United States
(12) Patent Application Publication
(10) Pub. No.: US 2026/0117188 A1
(43) Pub. Date: Apr. 30, 2026

(54) LIVER TISSUE-LIKE ORGANOID AND METHOD OF MANUFACTURING THE SAME

(71) Applicant: KOREA RESEARCH INSTITUTE OF BIOSCIENCE AND BIOTECHNOLOGY, Daejeon (KR)

(72) Inventors: Myung Jin SON, Daejeon (KR); Yongbo SHIN, Daejeon (KR); Seonju MUN, Daejeon (KR); Kyung-Sook CHUNG, Daejeon (KR); Cho-Rok JUNG, Daejeon (KR)

(21) Appl. No.: 18/925,174

(22) Filed: Oct. 24, 2024

(51) Int. Cl. C12N 5/071

(52) U.S. Cl. CPC C12N 5/0671; C12N 2500/25; C12N 2501/11; C12N 2501/115; C12N 2501/119; C12N 2501/12; C12N 2501/135; C12N 2501/155; C12N 2501/165; C12N 2501/22; C12N 2501/2303; C12N 2506/45; C12N 2513/00; C12N 2533/90

(57) ABSTRACT

The present invention relates to a method for preparing a liver tissue-like organoid (LTO) from a stem cell-derived liver organoid, and relates to a method for preparing LTO which comprises culturing the liver organoid in medium M1 comprising human bFGF, human VEGF-A, human PDGF-AB, human BMP4, human M-CSF and CHIR99021, and the liver tissue-like organoid (LTO) prepared by the method. The liver tissue-like organoid (LTO) of the present invention contains liver tissue-like biliary and vascular structures, as well as various hepatic cell types, including hepatocytes, cholangiocytes, hepatic stellate cells, endothelial cells, and immune cells, all of which maintain their specific functions. Therefore, the liver tissue-like organoid (LTO) can be usefully utilized in liver disease modeling, evaluation of toxicity and efficacy of drugs, and potential in vivo therapeutic applications.

### TECHNICAL FIELD

The present invention relates to a method for preparing liver tissue-like organoids (LTOs) from a stem cell-derived liver organoid, and liver tissue-like organoids (LTOs) prepared by the method.

### BACKGROUND ART

Organoids are used at the core of bioengineering technology that utilizes the self-organization ability of cells derived from test animals or humans to induce proliferation and utilize them as a living model system. As the technologies relating to organoids continue to develop, organoid models are being applied in various fields such as drug screening and toxicity evaluation.

Among them, the liver is a major organ with drug toxicity, and about 2 millions of people around the world die due to liver diseases every year; therefore, domestic and international market potential for liver organoids is high, and active studies on this are underway.

However, the liver is an organ formed by a complex intermingling of cells derived from the endoderm and the mesoderm. In particular, unlike other organs, the liver is composed of cells including hepatocytes, cholangiocytes, hepatic stellate cells, vascular endothelial cells and Kupffer cells, and there is complexity in that each cell has a different function and interacts with the others. In addition, even from the structural aspect, the liver is organized hierarchically down to lobules and individual cells, and functionally performs numerous functions such as metabolism, detoxification, and protein synthesis.

Therefore, in order to provide liver organoids that can be used to model liver diseases with high accuracy and to evaluate drug toxicity and efficacy, there is a complex challenge in that not only the various cell types present in the liver should be mimicked, but also the space and arrangement between the cells should be appropriately mimicked.

Accordingly, the inventors of the present invention have succeeded in developing a liver tissue-like organoid (LTO) that can be used for liver modeling, evaluation of toxicity and efficacy of drugs, etc. by having not only parenchymal cells and non-parenchymal cells expressed in the liver tissue, but also structures of blood vessels and a bile duct as well as structures specific to the liver, thereby completing the present invention.

### DISCLOSURE OF INVENTION

### Technical Problem

An object of the present invention is to provide a method for preparing a liver tissue-like organoid (LTO) that mimics the morphogenesis and developmental process of the liver, and a means that can be utilized for modeling liver diseases, and evaluating toxicity and efficacy of drugs, etc. by providing organoids prepared by the method.

### Solution to Problem

In order to achieve the above-identified object, an aspect of the present invention provides a method for preparing a liver tissue-like organoid (LTO) from a stem cell-derived liver organoid, comprising culturing the liver organoid in medium M1 comprising human bFGF, human VEGF-A, human PDGF-AB, human BMP4, human M-CSF and CHIR99021.

Another aspect of the present invention provides a liver tissue-like organoid (LTO) prepared by the method.

### Advantageous Effects of Invention

The liver tissue-like organoid (LTO) of the present invention has parenchymal cells and non-parenchymal cells expressed in the liver tissue, and particularly, has vascular and biliary structures specific to the liver tissue as well as the structures of hepatocytes, cholangiocytes, hepatic stellate cells, vascular endothelial cells, immune cells, etc.

As a result, the liver tissue-like organoid (LTO) of the present invention, which well mimics the morphogenesis and developmental process of the liver, has the effect of being useful for modeling liver diseases and evaluating the toxicity and efficacy of drugs with high accuracy.

### BRIEF DESCRIPTION OF DRAWINGS

FIG. 1 is a schematic diagram showing the process of preparing a liver organoid (LO) from induced pluripotent stem cells (iPSCs).

FIG. 2 is a schematic diagram showing the process of preparing a liver organoid (LO) from iPSCs to thereby prepare a liver tissue-like organoid (LTO) and a differentiation medium-liver organoid (DM-LO).

FIG. 3 shows microscopic images of the morphology and size of a liver tissue-like organoid (LTO) and a differentiation medium-liver organoid (DM-LO).

FIG. 4 shows graphs confirming the expression of hepatocyte markers (HNF4A and ALB) or a vascular endothelial cell marker (PEACAM1) in a liver tissue-like organoid (LTO) and a differentiation medium-liver organoid (DM-LO).

FIG. 5 shows images illustrating the morphology and size of a liver tissue-like organoid (LTO) prepared using different types of iPSCs (CRL2097, CW10175 and CW70203).

FIG. 6 shows graphs illustrating the expression levels of a hepatocyte marker (HNF4A), a cholangiocyte marker (KRT7) and a vascular endothelial cell marker (PECAM1) in a liver tissue-like organoid (LTO) prepared using iPSCs (CRL2097, CW10175 and CW70203).

FIG. 7 shows images illustrating the hepatocytes forming a bile canaliculi (BC) structure (markers: MRP2 and BSEP) and the cholangiocytes forming a bile duct (BD) structure (markers: KRT7 and KRT19), which were observed through immunostaining in a liver tissue-like organoid (LTO).

FIG. 8 shows images illustrating the hepatic stellate cells (markers: DESMIN and PDGFRB), vascular endothelial cells (markers: CD31 and LYVE1) and Kupffer cells (marker: MARCO), which were observed through immunostaining in a liver tissue-like organoid (LTO).

FIG. 9 shows images illustrating hepatocytes in the periportal region (marker: ASS1) and hepatocytes in the pericentral region (marker: GS), which were observed through immunostaining in a liver tissue-like organoid (LTO).

FIG. 10 shows images illustrating the structures of bile duct (BD) and vascular lumen (VL), which were observed through H&E staining in a liver tissue-like organoid (LTO).

FIG. 11 shows images illustrating the microvilli (MV) structure of the bile duct and fenestration of blood vessels (arrowheads) observed through TEM images in a liver tissue-like organoid (LTO) (bile canaliculi (BC); ductal lumen (DL); endothelial cells (EC); glycogen (Gly); hepatic cord (HC); nucleus (N); mitochondria (Mit); microvilli (Mv); and sinusoidal lumen (SL)).

FIG. 12 shows images illustrating the myeloid cells (monocytes, Kupffer cells, and granulocytes) and lymphoid cells (lymphocytes and NK cells) observed through Giemsa staining in a liver tissue-like organoid (LTO).

FIG. 13 shows images illustrating the myeloid immune cells (markers: CD11B and CD45) and lymphoid immune cells (markers: CD3 and CD56) observed through immunostaining in a liver tissue-like organoid (LTO).

FIG. 14 shows graphs illustrating the expression levels of immune cell markers (PTPRC, SPN and MARCO) observed at the RNA level in a liver tissue-like organoid (LTO) and LTO-derived suspension cells.

FIG. 15 shows graphs illustrating the results of confirming the functionality of immune cells by measuring the expression levels of inflammatory cytokines IL-1B, TNF-A and MCP1 in a liver tissue-like organoid (LTO).

FIG. 16 shows a graph illustrating the results of a 2.29-fold increase in serum protein synthesis ability (albumin synthesis ability) in a liver tissue-like organoid (LTO) compared to a differentiation medium-liver organoid (DM-LO). The liver tissue-like organoid (LTO) also exhibited a 1.81-fold increase in albumin secretion compared to the gold-standard primary human hepatocytes (PHHs).

FIG. 17 shows graphs illustrating the results of an increase in the activity of CYP3A4 (2.46-fold) and in the activity of CYP1A2 (3.77-fold), which are enzymes indicating drug metabolism activity, in a liver tissue-like organoid (LTO) compared to a differentiation medium-liver organoid (DM-LO).

FIG. 18 shows a graph illustrating the testosterone metabolic activity of a liver tissue-like organoid (LTO) (primary human hepatocytes (PHH); testosterone (TTT)).

FIG. 19 shows an image confirming glycogen storage ability by PAS staining (purple) and graphs confirming glucose metabolism ability, such as insulin responsiveness and gluconeogenesis, in a liver tissue-like organoid (LTO).

FIG. 20 shows images illustrating the results of confirming lipid metabolism ability (fat accumulation; Nile red) in a liver tissue-like organoid (LTO) (BSA: bovine serum albumin; FFA: free fatty acid; TG: triglyceride).

FIG. 21 shows images illustrating the results of confirming that dextran is well perfused into the liver through the blood vessels in a liver tissue-like organoid (LTO) compared to a differentiation medium-liver organoid (DM-LO).

FIG. 22 shows images illustrating the results of confirming that the bile duct structure and the vascular structure, which are formed in a liver tissue-like organoid (LTO), well maintain the functionality of the liver tissue that allows bile (CLF) and substance (Dextran) movement, as in the liver tissue in the body.

FIG. 23 shows images confirming that, when the toxicity of Busulfan was confirmed using a liver tissue-like organoid (LTO), damage to vascular endothelial cells (arrows) was observed from Day 2 of treatment with Busulfan, and the damage was gradually induced throughout the organoid up to Day 6.

FIG. 24 shows images confirming that, when a liver tissue-like organoid (LTO) was treated with Busulfan and acetaminophen (APAP) simultaneously, liver fibrosis due to liver damage progressed more rapidly than when treated with either Busulfan or APAP, respectively.

FIG. 25 shows images and graphs confirming that, when the efficacy of resmetirom was confirmed in a liver tissue-like organoid (LTO), the expression of the genes associated with fat accumulation and fat synthesis (DGAT2 and PLIN2) was decreased, the expression of the genes associated with activation of lipolysis (PNPLA2 and CPT1A) was increased, and the amount of triglycerides (TG) was decreased.

### BEST MODE FOR CARRYING OUT THE INVENTION

Hereinafter, the present invention will be described in detail.

### 1. Method for Preparing a Liver Tissue-Like Organoid (LTO)

In an aspect of the present invention provides a method for preparing a liver tissue-like organoid (LTO) from a stem cell-derived liver organoid, comprising culturing the liver organoid in medium M1 comprising human bFGF, human VEGF-A, human PDGF-AB, human BMP4, human M-CSF and CHIR99021.

In the present invention, the term “stem cell” refers to a cell that has self-renewal ability and is pluripotent or totipotent, that can differentiate into cells of all tissues of an organism. In a broad sense, the stem cell may also include embryoid bodies derived from embryonic stem cells.

In the present invention, the stem cell may include an embryonic stem cell of all origins, such as humans, monkeys, pigs, horses, cow, sheep, dogs, cats, mice, rabbits, etc., but are not limited thereto.

In the present invention, the stem cell may be a human embryonic stem cell (ESC) or human induced pluripotent stem cell (iPSC). The induced pluripotent stem cell refers to a cell induced to have pluripotent differentiation ability through an artificial dedifferentiation process from differentiated cells, and may be used as having the same meaning as “dedifferentiated stem cell”.

In the present invention, the term “organoid”, which is also called an organ mimic, refers to a three-dimensional cell aggregate formed through self-renewal and self-organization from an adult stem cell (ASC), an embryonic stem cell (ESC), and an induced pluripotent stem cell (iPSC). An organoid is a three-dimensional organ in vitro with a miniaturized, simplified form that mimics the anatomical structure of actual tissue, and it is possible to perform modeling of diseases based on a patient's genetic information, screening drugs through repeated tests by constructing organoids from the patient's tissue, etc.

The present inventors have prepared liver tissue-like organoids (LTO) from liver organoids derived from stem cells.

In the present invention, the term “liver tissue-like organoid (LTO)” refers to a structurally/functionally more developed form of organoid that can be prepared by further culturing the organoid in a medium having a special composition. More specifically, the liver tissue-like organoid (LTO) of the present invention has high liver functionality, has parenchymal cells (hepatocytes and cholangiocytes) and non-parenchymal cells (hepatic stellate cells, vascular endothelial cells, Kupffer cells and myeloid/lymphoid immune cells) expressed in the liver tissue, and particularly, has blood vessels (large vessels and capillaries) and biliary tract structures (structures of bile ducts and bile canaliculi) that are specific to liver tissue.

As such, a liver tissue-like organoid (LTO) of the present invention, which well mimics the morphogenesis and developmental process of the liver, can be usefully utilized for modeling liver diseases and evaluating the toxicity and efficacy of drugs with high accuracy.

In the present invention, the term “medium” refers to a medium suitable for supporting proliferation, survival and differentiation of a liver tissue-like organoid from a stem cell or liver organoids in vitro and refers to a mixture including nutrients necessary for the above-mentioned process. In particular, in the present invention, different types of media may be used depending on the culture stage of the liver tissue-like organoid.

Specifically, when preparing a liver tissue-like organoid (LTO) from a liver organoid in the present invention, hepatic medium (HM), expansion and mesodermal cell induction medium (EMM), differentiation and morphogenesis medium (DMM) and maturation medium (MM) are used sequentially. The specific compositions of these media are described in Table 1.

Meanwhile, in the present invention, for convenience purposes, media comprising different types and contents of nutrients may be referred to as media M1, M1a, M1b, M2, etc., but these media may not directly correspond to the above-mentioned HM, EMM, DMM or MM badges respectively.

Specifically, the medium MI may comprise human basic fibroblast growth factor (human bFGF), human VEGF-A, human PDGF-AB, human BMP4, human M-CSF, and CHIR99021 (Laduviglusib).

The medium M1a may further comprise, in addition to the composition of the M1 medium, human bFGF, human VEGF-A, human PDGF-AB, human BMP4, human M-CSF, CHIR99021, advanced DMEM/F12, P/S (penicillin/streptomycin), Glutamax, HEPES, N2 Supplement, B27 Supplement without Vitamin A, ITS, Nicotinamide, Forskolin, A83-01, [Leu]-gastrin I human, human HGF, human EGF, human BMP7, human FGF10 and human R-Spondin.

The medium M1b may further comprise, in addition to the composition of the M1 medium, human bFGF, human VEGF-A, human PDGF-AB, human BMP4, human M-CSF, CHIR99021, advanced DMEM/F12, P/S, Glutamax, HEPES, N2 Supplement, B27 Supplement with Vitamin A, A83-01, DAPT, dexamethasone, [Leu]-gastrin I human, human HGF, human EGF, human BMP7 and human FGF19.

The medium M2 may comprise William's E Medium, P/S, Glutamax, HEPES, N2 Supplement, B27 Supplement with Vitamin A, Knockout SR, A83-01, DAPT, dexamethasone, Y-27632 and human IL-3.

An aspect of the present invention provides a method for preparing a liver tissue-like organoid (LTO) from a stem cell-derived liver organoid, comprising culturing the liver organoid in medium M1 comprising human bFGF, human VEGF-A, human PDGF-AB, human BMP4, human M-CSF, and CHIR99021.

Another aspect of the present invention provides a method for preparing LTO, further comprising culturing the liver organoid in medium M2 comprising William's E Medium, P/S, Glutamax, HEPES, N2 Supplement, B27 Supplement with Vitamin A, Knockout SR, A83-01, DAPT, dexamethasone, Y-27632 and human IL-3.

Still another aspect of the present invention provides a method for LTO, wherein in culturing in the medium M1, the medium M1 further comprises any one or more selected from the group consisting of advanced DMEM/F12, William's E Medium, P/S, Glutamax, HEPES, N2 Supplement, B27 Supplement without Vitamin A, B27 Supplement with Vitamin A, ITS, Knockout SR, Nicotinamide, N-acetylcysteine, Forskolin, A83-01, DAPT, Dexamethasone, Y-27632, [Leu]-gastrin I human, Oncostatin M, human HGF, human EGF, human BMP7, human FGF10, human FGF19 and human R-Spondin.

Still another aspect of the present invention provides a method for LTO, wherein the culturing in the medium MI further comprises culturing in medium M1a, which further comprises human bFGF, human VEGF-A, human PDGF-AB, human BMP4, human M-CSF, CHIR99021, Advanced DMEM/F12, P/S, Glutamax, HEPES, N2 Supplement, B27 Supplement without Vitamin A, ITS, Nicotinamide, Forskolin, A83-01, [Leu]-gastrin I human, human HGF, human EGF, human BMP7, human FGF10 and human R-Spondin; and culturing in medium M1b, which further comprises human bFGF, human VEGF-A, human PDGF-AB, human BMP4, human M-CSF, CHIR99021, Advanced DMEM/F12, P/S, Glutamax, HEPES, N2 Supplement, B27 Supplement with Vitamin A, A83-01, DAPT, Dexamethasone, [Leu]-gastrin I human, human HGF, human EGF, human BMP7 and human FGF19.

Still another aspect of the present invention provides a method for LTO, which, when the time point at which a liver organoids derived from a stem cell is prepared into a liver tissue-like organoid (LTO) is set as Day 0 (DO), comprising (i) culturing the stem cell-derived liver organoid in suspension in a medium comprising extracellular matrix (ECM) from Day 0 (DO) to Day 4 (D4); (ii) culturing the culture product obtained in step (i) in a state being embedded in the ECM from Day 4 (D4) to Day 12 (D12); and (iii) culturing the culture product obtained in step (ii) under flow conditions from Day 12 (D12) to Day 16 (D16).

The ECM may be Matrigel®, and in the suspension culture step, may mean 3% Matrigel®.

Specifically, the method of the present invention may comprise the steps of culturing the liver organoid (i) in suspension in HM medium comprising ECM for a period of from Day 0 (DO) to Day 1 (D1); (ii) in suspension in EMM medium comprising ECM for a period of from Day 1 (D1) to Day 4 (D4); (iii) in DMM medium in a state being embedded in the ECM for a period of from Day 4 (D4) to Day 10 (D10); and in MM medium in a state being embedded in the ECM for a period of from Day 10 (D10) to Day 12 (D12); and (iv) under flow conditions for a period of from Day 12 (D12) to Day 16 (D16).

In the present invention, the term “under flow conditions” indicate applying shear stress or mechanical force to a cell or an organoid in order to closely mimic in-vivo conditions, and may include culturing while rocking the medium, but is not limited thereto. In the present invention, the M1a, M1b and M2 media may each refer to EMM, DMM and MM media. The meanings and compositions of the HM, EMM, DMM and MM media are described in Example 2 and [Table 1] below.

Still another aspect of the present invention provides a method for preparing LTO, in which the liver organoid is derived from a stem cell by a culture method, which comprises differentiating the stem cell into a definitive endoderm (DE) cell; and differentiating the definitive endoderm cell into a hepatic endoderm (HE) cell.

The step of differentiating a stem cell into a definitive endoderm (DE) cell comprises separating the stem cell by treating with ReLeSR (STEMCELL Technologies), plating at a 1:50 rate on diluted Matrigel coated plates with a confluency of 70-80%, culturing in mTesR medium (STEMCELL Technologies) for 1 to 2 days, and culturing for 4-5 days in RPMI 1640 medium (Thermo Fisher) supplemented with 1×B27 without insulin (Thermo Fisher) and 100 ng/ml activin A (PeproTech).

The step of differentiating the definitive endoderm cell into a hepatic endoderm (HE) cell comprises replacing the medium with RPMI 1640 medium, in which 1×B27 (Thermo Fisher), 10 ng/mL of basic fibroblast growth factor (bFGF, PeproTech), and 20 ng/ml of bone morphogenetic protein 4 (BMP4, PeproTech) are added, and culturing for 4 days under a 5% O₂ hypoxic condition.

### 2. Liver Tissue-Like Organoid (LTO)

An aspect of the present invention provides a liver tissue-like organoid (LTO).

The liver tissue-like organoid (LTO) of the present invention may be prepared by the method described in the “1. Method for preparing a liver tissue-like organoid (LTO)” above, and those described in the item above apply in the same way.

The liver tissue-like organoid (LTO) of the present invention relates to a highly functional organoid, which has various types of parenchymal cells and non-parenchymal cells present in the liver tissue and structures of a bile duct and blood vessels specific to the liver tissue, by way of well mimicking the morphogenesis and development process of the liver, and thus can be applied to modeling of liver diseases and evaluation of toxicity and efficacy of drugs with high accuracy.

In another aspect of the present invention, the liver tissue-like organoid (LTO) expresses MRP2, BSEP, KRT7, KRT19, DESMIN, PDGFRB, CD31(PEACAM), LYVE1 and MARCO markers.

In another aspect of the present invention, the liver tissue-like organoid (LTO) further expresses one or more markers selected from the group consisting of HNF4A, ALB, ASS1, GS, PTPRC, SPN, CD11B, CD3, and CD56.

In still another aspect of the present invention, in the liver tissue-like organoid (LTO), development of hepatocytes, cholangiocytes, hepatic stellate cells, vascular endothelial cells and immune cells is observed. In the present invention, immune cells may include Kupffer cells but is not limited thereto.

In the present invention, the term “development” means that cells, tissues, organs, etc. are fully developed and well matured to be able to perform functions thereof.

Still another aspect of the present invention provides a liver tissue-like organoid (LTO), wherein the liver tissue-like organoid (LTO) has a bile duct and a blood vessel structure, and in which bile can move through the bile duct and substance can move through the blood vessels.

The liver tissue-like organoid (LTO) of the present invention, in which all of various types of parenchymal cells and non-parenchymal cells present in the liver tissue, a bile duct and blood vessels specific to the liver tissue, and even immune cells are developed, provides an organoid model similar to a highly-functional liver tissue.

Hereinafter, the present invention will be described in more detail through examples. These examples are only intended to illustrate the present invention, and the content of the present invention is not limited by these examples.

### Example 1. Preparation of Liver Organoid (LO) Using Induced Pluripotent Stem Cells (iPSCs)

Preparation of iPSCs

iPSCs (CRL2097), which were prepared by reprogramming human foreskin fibroblasts, and iPSCs (CW10175 and CW70203), which were purchased from FUJIFILM Cellular Dynamics, were cultured under feeder cell-free conditions.

Specifically, human foreskin fibroblasts (HFF) (CRL-2097) purchased from the American Type Culture Collection (ATCC) were seeded in 6-well plates at 2× 10⁵ cells/well, and on Day 2, the cells were transduced with Sendai virus using CytoTune®-iPS 2.0 Sendai Reprogramming Kit (Thermo Fisher; A16517). On Day 3, the medium was replaced with fresh fibroblast culture medium (DMEM including 10% fetal bovine serum, 1% NEAA, 1 mM L-glutamine and 0.1 mM β-mercaptoethanol), and then, on Day 9, 1×10⁵ cells/well were transferred to 6-well plates coated with 2% Matrigel and cultured under feeder-free conditions. On the following day, the medium was replaced with PSC medium, and was replaced daily with a fresh medium. iPSC colonies were selected on about Day 22 of reprogramming.

Thereafter, the iPSCs were cultured for 7 days in mTeSR1 medium (STEMCELL Technologies) on Matrigel (Corning)-coated dishes diluted 1:100 in a 5% CO₂ incubator, and subcultured every week. Additionally, mycoplasma contamination was tested once a month using the EZ-PCR Mycoplasma Detection Kit (Biological Industries).

### Preparation and Culture of Liver Organoids

In order to differentiate iPSCs into definitive endoderm (DE) cells, PSCs were decomposed by treating with 1 mL of ReLeSR (STEMCELL Technologies) per a 35 mm culture dish for 5 minutes or more, the cells were plated with a confluency of 70-80% on Matrigel-coated plates diluted 1:50 and cultured in mTeSR1 medium (STEMCELL Technologies) for 1-2 days.

Then, iPSCs with a confluency of 90% were cultured in RPMI 1640 medium (Thermo Fisher) supplemented with 1×B27 without insulin (Thermo Fisher) and 100 ng/ml activin A (PeproTech) for 4-5 days.

In order to differentiate complete endoderm cells into hepatic endoderm (HE) cells, the medium was replaced with RPMI 1640 supplemented with 1×B27 (Thermo Fisher), 10 ng/ml of basic fibroblast growth factor (bFGF, PeproTech) and 20 ng/ml of bone morphogenetic protein 4 (BMP4, PeproTech), and cultured under hypoxic conditions of 5% O₂ for 4 days.

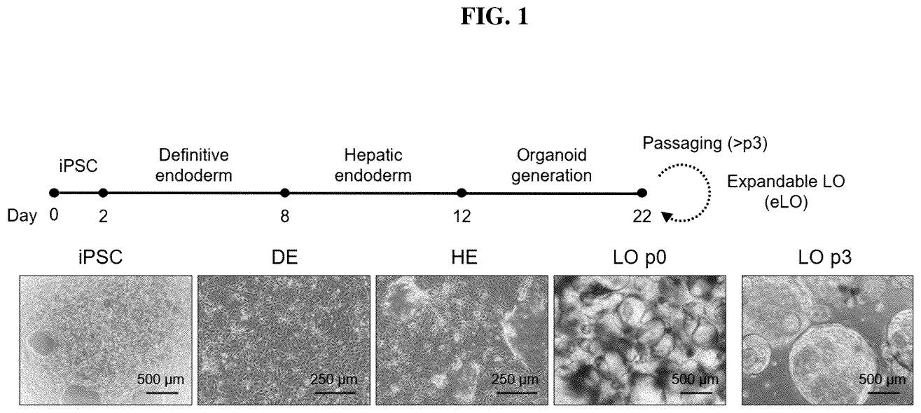

Thereafter, in order to form three-dimensional (3D) organoids, hepatic endoderm cells were separated into single cells using TrypLE Express Enzyme (Thermo Fisher), so that 100,000 cells were included in a 30 μL of a Matrigel drop. The cells were cultured in hepatic medium (HM) with 10 UM of the ROCK inhibitor Y-27632 (Tocris) for 3 days to improve cell viability. Then, the medium was replaced with HM medium without Y-27632 and cultured at 37° C. in a 5% CO₂ environment for 7 days, with the medium replaced every 2 days (FIG. 1).

The proliferated liver organoids (LOs) were mechanically separated every 7 days using a surgical blade under a microscope at a ratio of 1:3 to 1:10. For long-term storage, subcultured liver organoids (LO) were cryopreserved using the CryoStor (STEMCELL Technologies).

### Example 2. Preparation of Liver Tissue-Like Organoid (LTO) from Liver Organoid (LO)

In order to further differentiate the liver organoids prepared in Example 1 above and to induce morphogenesis and vascularization, the mechanically separated liver organoids were suspended in hepatic medium (HM) including 3% Matrigel (Day 0, DO). A suspension of liver organoids was plated onto 6-well flat bottom ultra-low attachment plates (Corning) and cultured for one day (Day 1, D1).

Then, the medium was replaced with expansion and mesodermal cell induction medium (EMM) including 3% Matrigel and cultured under a suspended condition for additional 3 days, and on Day 4 (D4), the liver organoids cultured in this way were embedded in Matrigel, and cultured for 6 days in differentiation and morphogenesis medium (DMM) to thereby prepare vascularized liver tissue-like organoids (LTO).

On Day 10 (D10), in order to promote functional maturation of vascularized liver tissue-like organoids, the medium was replaced with maturation medium (MM) including Y-27632 and cultured for additional 2 days (D12) while retaining the state where the organoids are embedded in Matrigel. Then, the liver tissue-like organoids were cultured using a bi-directional rocker in MM medium for 4 days or more, and the liver tissue-like organoids were maintained at 37° C. in a 5% CO₂ environment, thereby completing the liver tissue-like organoids (LTOs).

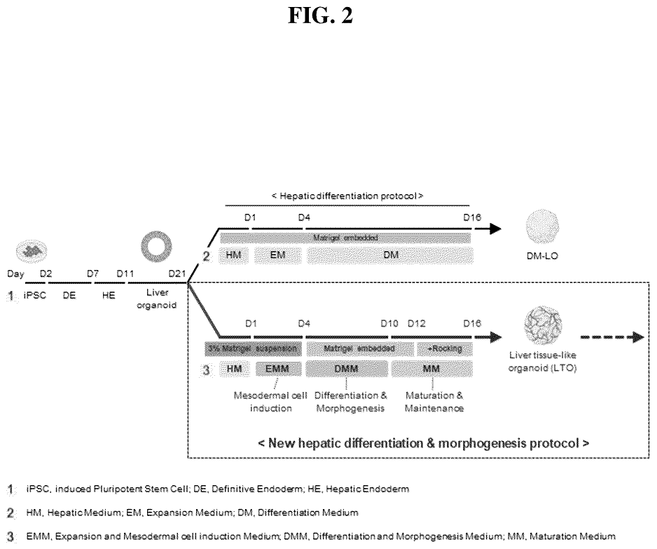

Such a preparation process of the liver tissue-like organoids (LTOs) is illustrated in FIG. 2, and the compositions of the HM, EMM, DMM and MM media used in Examples 1 and 2 above are as described in Table 1 below.

TABLE 1

| | | HM | | EMM | | DMM | | MM | |
|---|---|---|---|---|---|---|---|---|---|
| Reagent | Company | Working Concentration | | | | | | | |
| Advanced | Thermo Fisher | 1X | | 1X | | 1X | | | |
| DMEM/F12 | | | | | | | | | |
| William's E | Thermo Fisher | | | | | | | 1X | |
| Medium | | | | | | | | | |
| Penicillin/Streptomycin | Thermo Fisher | 1% | | 1% | | 1% | | 1% | |
| (P/S) | | | | | | | | | |
| Glutamax | Thermo Fisher | 1% | | 1% | | 1% | | 1% | |
| HEPES | Thermo Fisher | 1% | | 1% | | 1% | | 1% | |
| N2 Supplement | Thermo Fisher | 1X | | 1X | | 1X | | 1X | |
| B27 Supplement | Thermo Fisher | 1X | | 1X | | | | | |
| without Vitamin A | | | | | | | | | |
| B27 Supplement | Thermo Fisher | | | | | 1X | | 1X | |
| with Vitamin A | | | | | | | | | |
| Insulin-Transferrin- | Thermo Fisher | 1X | | 1X | | | | | |
| Selenium (ITS) | | | | | | | | | |
| Knockout SR | Thermo Fisher | | | | | | | 1% | |
| Nicotinamide | Sigma-Aldrich | 10 | mM | 10 | mM | | | | |
| N-Acetylcysteine | Sigma-Aldrich | 1 | mM | | | | | | |
| Forskolin | Sigma-Aldrich | 10 | μM | 10 | μM | | | | |
| A83-01 | Tocris | 5 | μM | 0.5 | μM | 0.5 | μM | 0.5 | μM |

| | | | | | | | |
|---|---|---|---|---|---|---|---|
| DAPT | Sigma-Aldrich | | | 5 | μM | 5 | μM |
| Dexamethasone | Sigma-Aldrich | 0.1 | μM | 3 | μM | 0.1 | μM |

| Y-27632 | Tocris | | | | | | | 10 μM |
|---|---|---|---|---|---|---|---|---|
| | | | | | | | | (for 2 d) |
| [Leu]-Gastrin I | Sigma-Aldrich | 10 | nM | 10 | nM | 10 | nM | |
| human | | | | | | | | |
| Oncostatin M | R&D | 10 | ng/mL | | | | | |
| Human HGF | Peprotech | 25 | ng/mL | 25 | ng/mL | 25 | ng/mL | |
| Human EGF | Peprotech | 50 | ng/mL | 50 | ng/mL | 50 | ng/mL | |
| Human BMP7 | Peprotech | | | 25 | ng/mL | 25 | ng/mL | |
| Human FGF10 | Peprotech | | | 100 | ng/mL | | | |
| Human FGF19 | Peprotech | | | | | 100 | ng/mL | |
| Human R-spondin | R&D | | | 1 | μg/mL | | | |
| Human bFGF | Peprotech | 10 | ng/mL | 30 | ng/mL | 30 | ng/mL | |

| | | | | | |
|---|---|---|---|---|---|
| Human VEGF-A | Peprotech | 10 | ng/mL | 10 | ng/mL |
| Human PDGF-AB | Peprotech | 10 | ng/mL | 10 | ng/mL |
| Human BMP4 | Peprotech | 20 | ng/mL | 20 | ng/mL |

| Human M-CSF | Peprotech | 50 | ng/mL | 50 ng/mL |
|---|---|---|---|---|
| | | | | (for 2 d) |
| CHIR99021 | Tocris | 3 | μM | 3 μM |

| | | (for 2 d) | | |
|---|---|---|---|---|
| Human IL-3 | Peprotech | | 10 | ng/mL |

### Comparative Example 1. Preparation of Differentiation Medium-Liver Organoid (DM-LO)

The liver organoids prepared in Example 1 above were cultured in hepatic medium (HM) for 1 day, in expansion medium (EM) for 3 days and in differentiation medium (DM) for 12 days to thereby prepare differentiation medium-liver organoids (DM-LOs).

The process of preparing differentiation medium-liver organoids (DM-LOs) is illustrated in FIG. 2, and the compositions of the HM, EM and DM media used here are as described in Table 2 below.

TABLE 2

| | | HM | | EM | | DM | |
|---|---|---|---|---|---|---|---|
| Reagent | Company | Working Concentration | | | | | |
| Advanced | Thermo Fisher | 1X | | 1X | | 1X | |
| DMEM/F12 | | | | | | | |
| Penicillin/Streptomycin | Thermo Fisher | 1% | | 1% | | 1% | |
| (P/S) | | | | | | | |
| Glutamax | Thermo Fisher | 1% | | 1% | | 1% | |
| HEPES | Thermo Fisher | 1% | | 1% | | 1% | |
| N2 Supplement | Thermo Fisher | 1X | | 1X | | 1X | |
| B27 Supplement | Thermo Fisher | 1X | | 1X | | | |
| without Vitamin A | | | | | | | |
| B27 Supplement | Thermo Fisher | | | | | 1X | |
| with Vitamin A | | | | | | | |
| Insulin-Transferrin- | Thermo Fisher | 1X | | | | | |
| Selenium (ITS) | | | | | | | |
| Nicotinamide | Sigma-Aldrich | 10 | mM | 10 | mM | | |
| N-Acetylcysteine | Sigma-Aldrich | 1 | mM | 1 | mM | 1 | mM |
| Forskolin | Sigma-Aldrich | 10 | μM | 10 | μM | | |
| A83-01 | Tocris | 5 | μM | 5 | μM | 0.5 | μM |
| DAPT | Sigma-Aldrich | | | | | 10 | μM |
| Dexamethasone | Sigma-Aldrich | 0.1 | μM | | | 3 | μM |
| Human bFGF | Peprotech | 10 | ng/mL | | | | |
| [Leu]-Gastrin I | Sigma-Aldrich | 10 | nM | 10 | nM | 10 | nM |
| human | | | | | | | |
| Oncostatin M | R&D | 10 | ng/mL | | | | |
| Human HGF | Peprotech | 25 | ng/mL | 25 | ng/mL | 25 | ng/mL |
| Human EGF | Peprotech | 50 | ng/mL | 50 | ng/mL | 50 | ng/mL |

| | | | | | |
|---|---|---|---|---|---|
| Human BMP7 | Peprotech | 25 | ng/mL | 25 | ng/mL |

| | | | |
|---|---|---|---|
| Human FGF10 | Peprotech | 100 | ng/mL |

| | | | |
|---|---|---|---|
| Human FGF19 | Peprotech | 100 | ng/mL |

| | | | |
|---|---|---|---|
| Human R-spondin | R&D | 1 | μg/mL |

### Experimental Example 1. Characterization of Liver Tissue-Like Organoids (LTOs)

### Experimental Example 1.1. Morphology and Size of Liver Tissue-Like Organoids (LTOs)

### Observation of Morphology and Size Through Microscope

The morphology and size of the liver tissue-like organoids (LTOs) prepared in

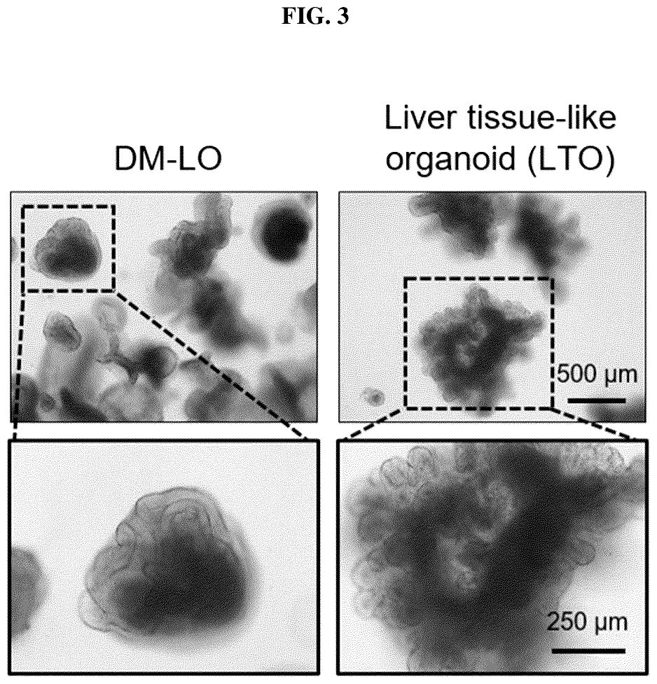

Example and the differentiation medium-liver organoids (DM-LOs) prepared in Comparative Example were compared. In the case of the liver tissue-like organoids (LTOs), the size was increased compared to the existing differentiation medium-liver organoids (DM-LOs), and a more complex structure was observed inside and outside the organoids (FIG. 3).

### Confirmation of Presence of Expression of Liver Morphology Markers

During the differentiation process of liver tissue-like organoids (LTOs), it was confirmed that the expression of HNF4A and ALB, which are hepatocyte markers indicating liver functions, was increased as differentiation progressed, and that the expression of PECAM1, which is a vascular endothelial cell marker necessary for vascularization, was also increased together.

In particular, it was confirmed that the expression of markers of hepatocytes and vascular endothelial cells was significantly increased after using the growth factor-free maturation medium (MM), which was used in the Examples above (D10 to D16).

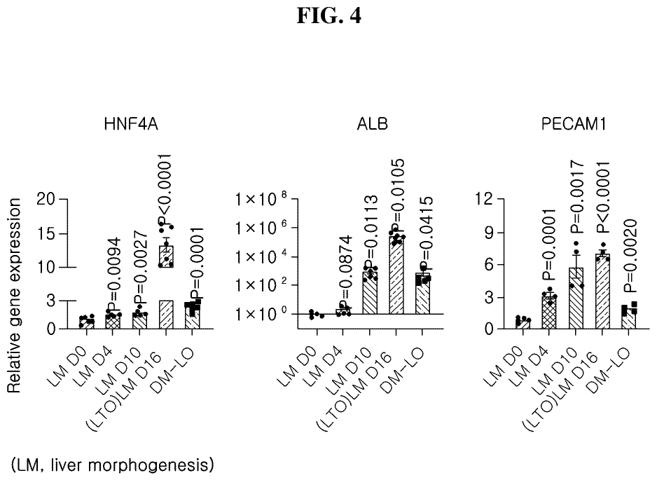

In contrast, in the case of the differentiation medium-liver organoids (DM-LOs), it was confirmed that the expression levels of the markers were lower than those of the liver tissue-like organoids (LTOs), in the case of liver markers in liver tissue-like organoids (LTOs), HNF4A was increased 5.7-fold and ALB was increased 387-fold or higher compared to those of differentiation medium-liver organoids (DM-LOs), and in the case of vascular endothelial cells, it was confirmed that PEACAM1 was increased 4.3-fold or higher (FIG. 4).

Confirmation of Differentiation into Liver Tissue-Like Organoids (LTOs) Using Different Types of iPSCs (CRL2097, CW10175 and CW70203)

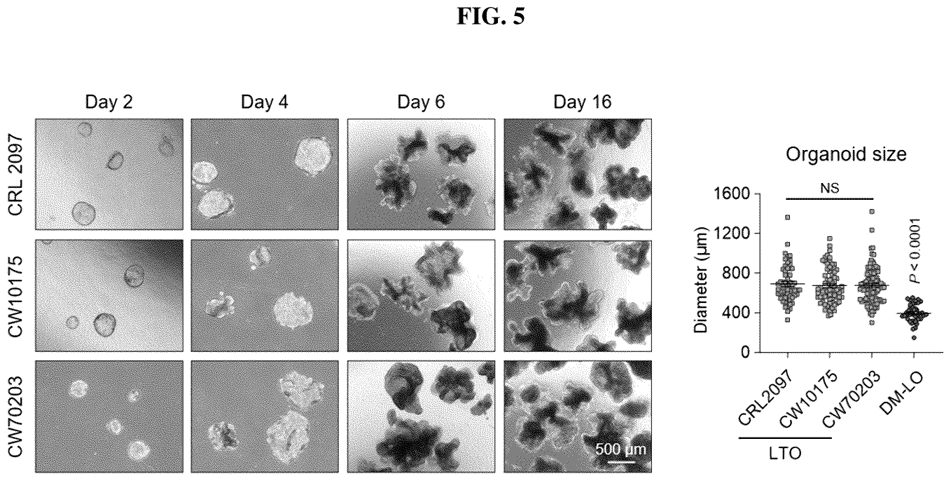

In order to determine whether the liver tissue-like organoids (LTOs) are differentiated only from a specific type of iPSCs, LTOs were prepared in the same manner using CRL2097, CW10175 and CW70203, which are iPSCs from different individuals, and even in this case, a more complex structure was observed inside and outside of the LTOs compared to the differentiation medium-liver organoids (DM-LOs). In addition, it was confirmed that these LTOs were prepared with a relatively constant size (690.5+212.1 μm) and shape regardless of the type of iPSCs (FIG. 5).

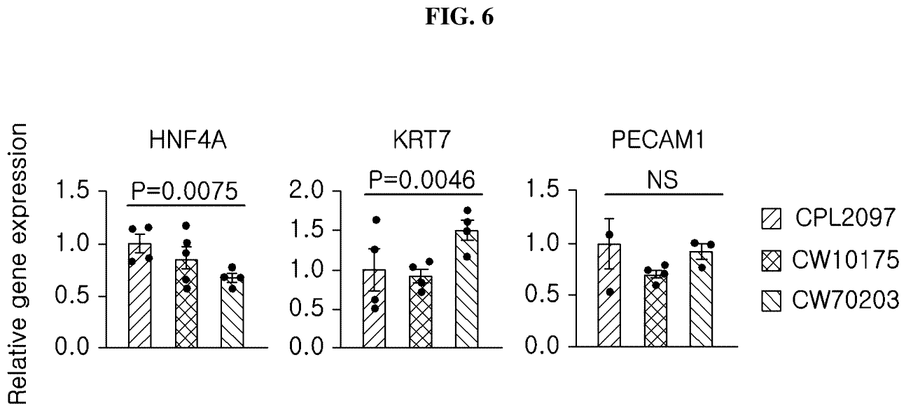

Furthermore, it was confirmed that hepatocyte markers (HNF4A), cholangiocyte markers (KRT7) and vascular endothelial cell markers (PECAM1) were similarly expressed at similar levels in each organoid (FIG. 6).

As described above, as a result of examining the characteristics of the liver tissue-like organoids (LTOs) of the present invention, it was confirmed that it is possible to prepare organoids with low batch variation and high reproducibility.

### Experimental Example 1.2. Structure of Liver Tissue-Like Organoids (LTOs)

### Immunostaining to Examine the Structure of Liver Tissue

In order to confirm whether the characteristic of liver tissue structure is well formed in the liver tissue-like organoids (LTOs), immunostaining was performed with markers of hepatocytes, cholangiocytes, hepatic stellate cells, vascular endothelial cells and Kupffer cells present in liver tissue.

The markers for each of these cells are as described in Table 3 below.

TABLE 3

| Cell Type | Markers |
|---|---|
| Hepatocytes | MRP2, BSEP |
| Cholangiocytes | KRT7, KRT19 |
| Hepatic stellate cells | DESMIN, PDGFRB |
| Vascular endothelial cells | CD31, LYVE1 |
| Kupffer cells | MARCO |

### Observation of Liver Tissue Structure Through Immunostaining

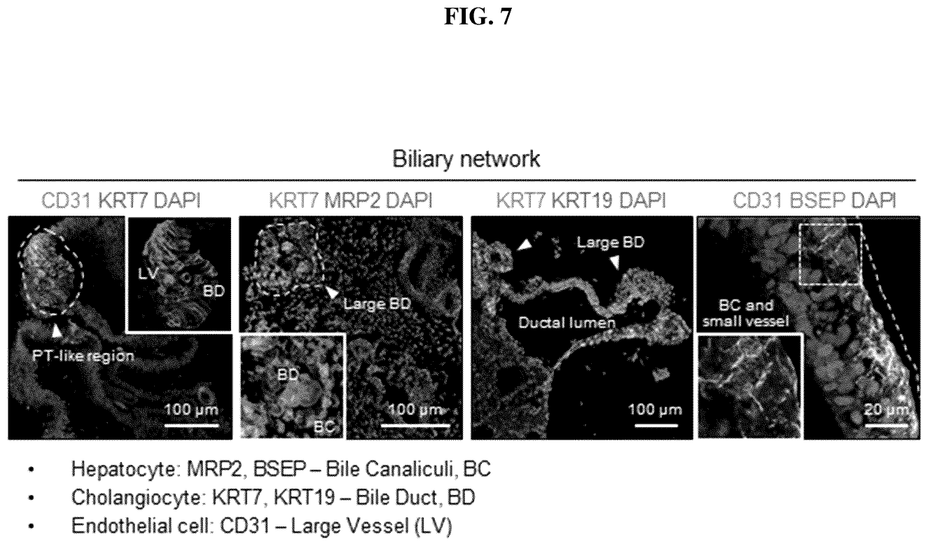

In this case, hepatocytes stained with BSEP and bile canaliculi (BC) formed there between were clearly observed, and the presence of bile ducts (BD) stained with KRT7 and KRT19 was confirmed. In particular, it was confirmed that blood vessels stained with CD31 and bile ducts stained with KRT7 were each formed in the form of portal triads on the external side of the organoids (FIG. 7).

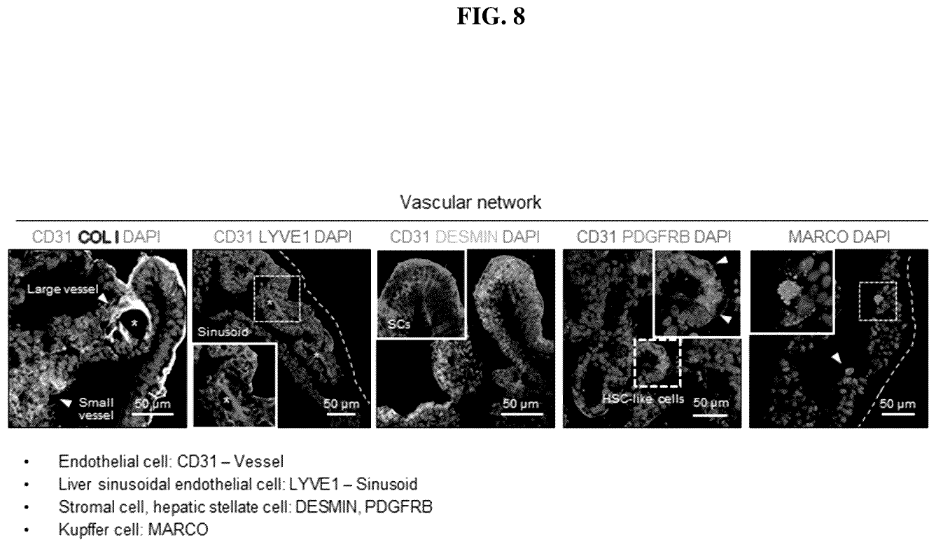

In addition, it was observed that sinusoidal vascular endothelial cells, which are liver-specific capillaries stained with LYVE1, as well as large blood vessels stained with CD31, were well distributed forming a vascular network (FIG. 8).

In addition, it was confirmed that hepatic stellate cells stained with DESMIN and PDGFRB were distributed around blood vessels and that extracellular matrix such as collagen I was well distributed around blood vessels, thereby confirming a structure similar to that of actual liver tissue. In addition, the presence of Kupffer cells, which are the liver tissue resident macrophages (MARCO staining), was confirmed (FIG. 8).

Meanwhile, the liver tissue in the body is has a form where a small unit structure in the form of a lobule is repeated, and the hepatic zonation is divided into zones 1, 2 and 3 based on the distance from the portal vein (PV) to the central vein (CV), and the function of the hepatocytes varies depending on the location.

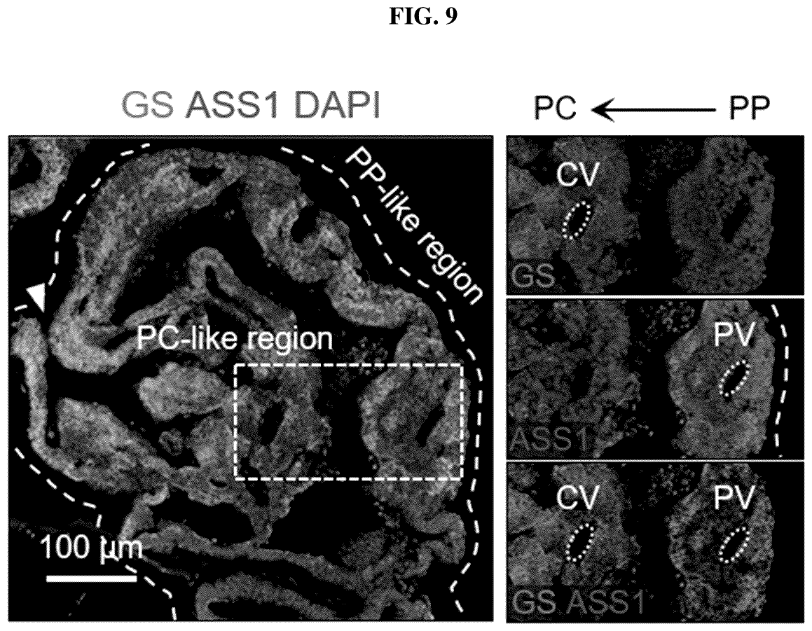

It was confirmed in the liver tissue-like organoids (LTOs) prepared in the Examples above that from the external side to the internal side of the organoids, the expression of ASS1 (which is a hepatocyte marker in the periportal region (i.e., zones 1 and 2)) and GS (which is a hepatocyte marker in the pericentral region (i.e., zone 3)) was distinctly shown, in a manner similar to the liver zonation phenomenon observed in the lobules of liver tissue in the body (FIG. 9).

Since the metabolic function of hepatocytes varies depending on the liver zonation, it is determined that more accurate drug evaluation will be possible by utilizing liver tissue-like organoids (LTOs) with high structural and functional maturity.

### Observation of Liver Tissue Structure Through H&E Staining

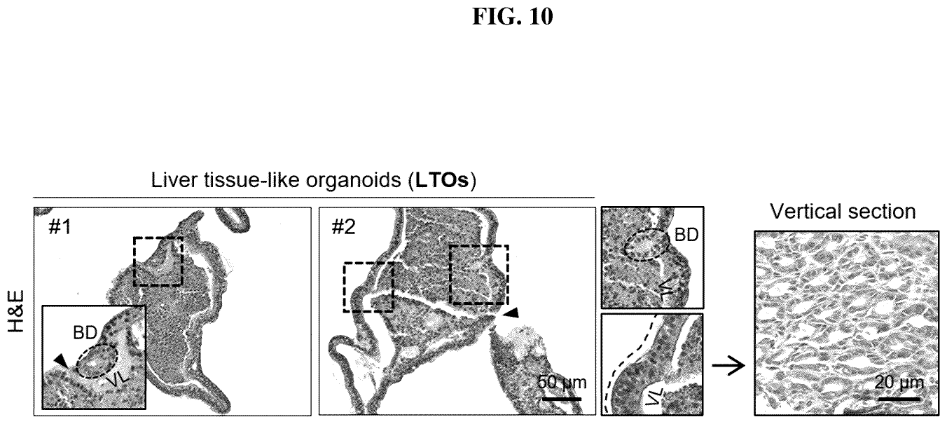

Likewise, even in H&E staining images, it was possible to confirm the structures of bile canaliculi (BC) and bile duct (BD) in the liver tissue-like organoids (LTOs) in which morphogenesis and vascularization were induced, and it was possible to confirm that the vascular lumen (VL) was well formed throughout the organoids (FIG. 10).

### Observation of Liver Tissue Structure Through TEM

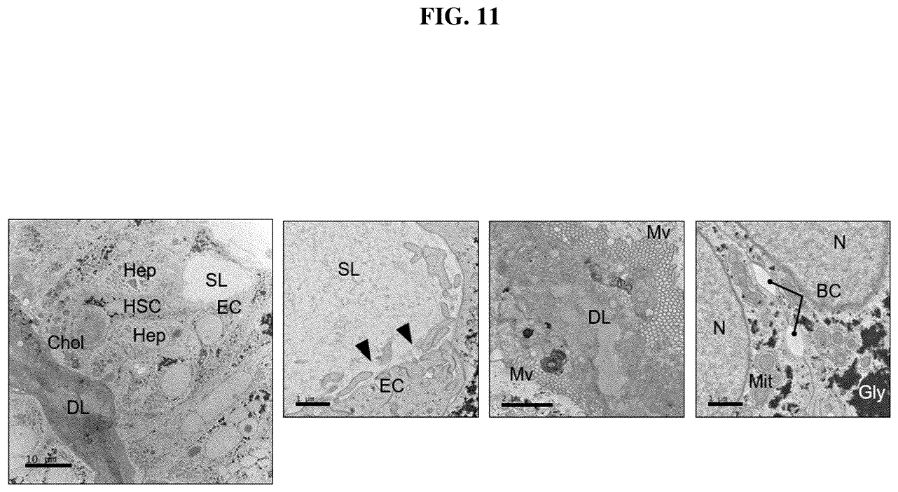

Through TEM images, it was observed that the microvilli (MV) structure was well developed in the direction of the ductal lumen (DL) of the bile duct in the liver tissue-like organoids (LTOs), thereby confirming that the characteristics of the bile duct in the liver were well reflected (FIG. 11).

In particular, it was confirmed that fenestration, which is a characteristic of liver-specific sinusoids that are formed to facilitate the movement of substances in blood vessels present in the liver, was well formed in the blood vessels inside the organoids (FIG. 11, arrowheads).

### Experimental Example 1.3. Cell Compositions of Liver Tissue-Like Organoids (LTOs)

It was confirmed that liver tissue-like organoids (LTOs) include not only parenchymal cells (e.g., hepatocytes and cholangiocytes), but also non-parenchymal cells (e.g., hepatic stellate cells and endothelial cells), and in particular, various types of immune cells, including Kupffer cells, exist.

### Observation Through Giemsa Staining

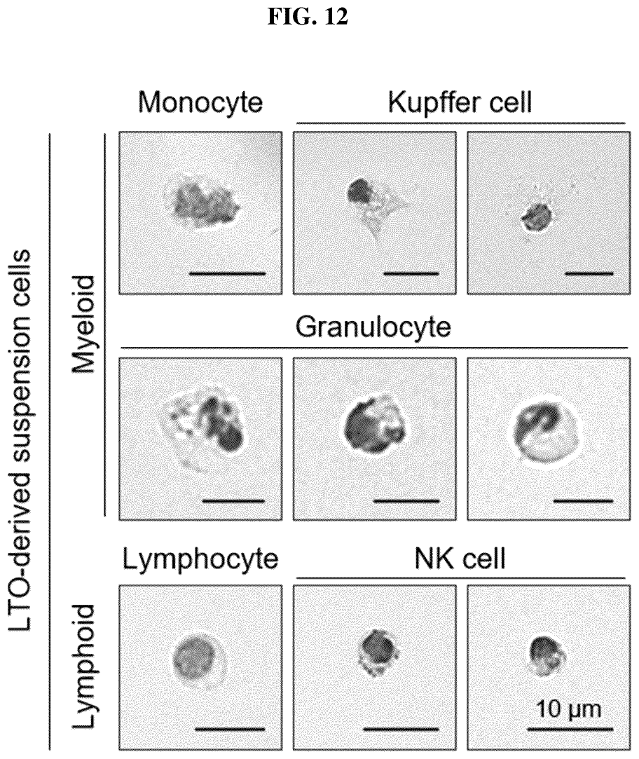

First, it was confirmed in the morphological analysis using Giemsa staining that myeloid cells (e.g., monocytes, Kupffer cells and granulocytes) and lymphoid cells (e.g., lymphocytes and NK cells) were observed (FIG. 12).

### Observation Through Immunostaining

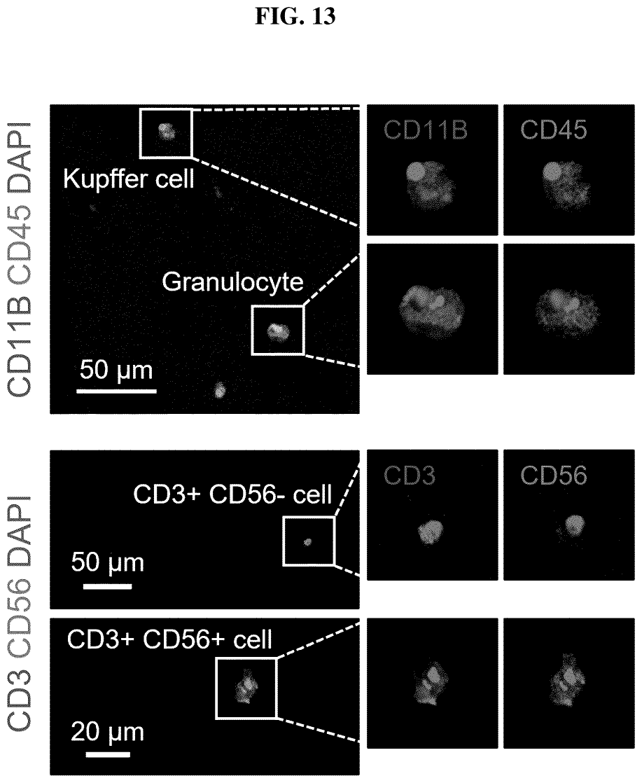

In addition, myeloid immune cells, which are stained with CD11B and CD45, and lymphoid immune cells, which are stained with CD3 and CD56, were identified through immunostaining (FIG. 13).

### Observation at the RNA Level

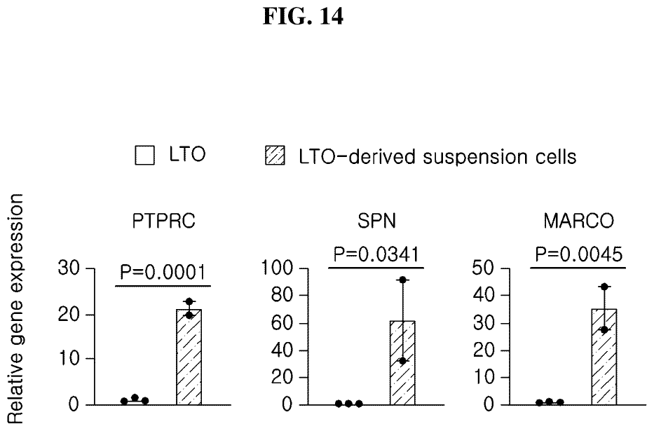

It was confirmed that representative immune cell markers (PTPRC, SPN and MARCO) were well expressed even at the RNA level (FIG. 14).

Specifically, it was confirmed through the above markers that liver tissue-like organoids (LTOs) include various types of myeloid/lymphoid immune cells, including Kupffer cells.

### Experimental Example 1.4. Confirmation of Functionality of Liver Tissue-Like Organoids (LTOs)

### Confirmation of Functionality of Immune Cells

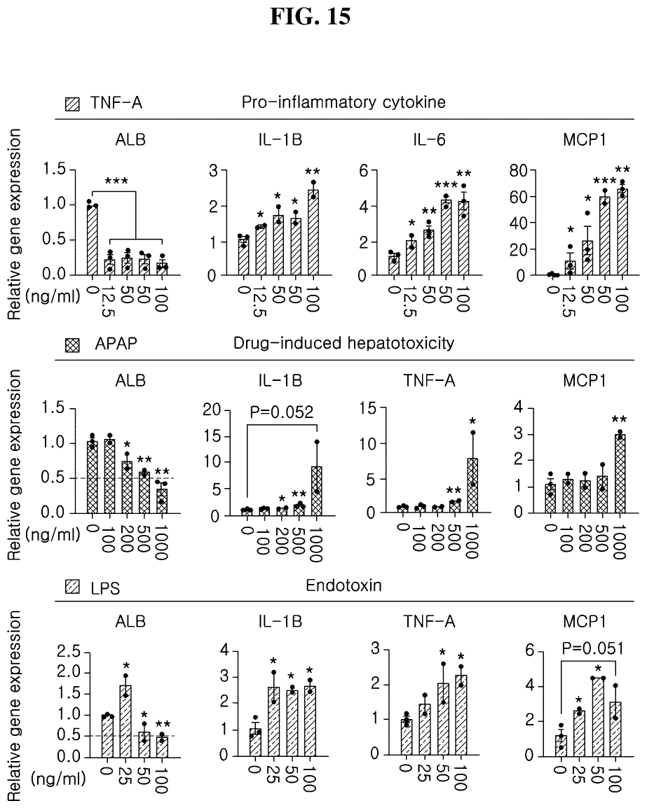

In order to confirm the functionality of immune cells present in liver tissue-like organoids (LTOs), the responses to inflammatory cytokines (TNF-A), lipopolysaccharides (LPSs) (i.e., pathogen-associated molecular patterns (PAMPs)), and acetaminophen (APAP)-induced hepatocyte death (i.e., a damage-associated molecular pattern (DAMP)) were confirmed (FIG. 15).

In particular, it was confirmed that the expression of IL-1B, TNF-A and MCP1, which are immune cell-specific inflammatory cytokines, were increased in each treatment group, thereby confirming that the immune reactivity (functionality) of various immune cells present in the liver tissue-like organoids (LTOs) was well maintained (FIG. 15).

### Confirmation of Function of Synthesizing Serum Protein

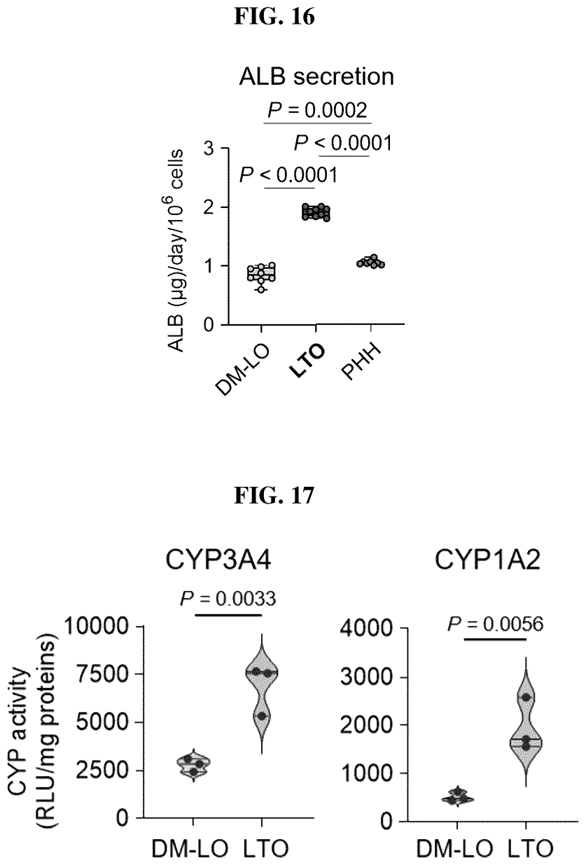

The ability of serum protein synthesis (albumin synthesis ability) (i.e., a representative liver function) was confirmed using human ALB ELISA Kit (Bethyl Laboratories) in the liver tissue-like organoids (LTOs). It was confirmed that the amount of albumin (ALB) synthesis was increased 2.29-fold and 1.81-fold compared to the differentiation medium-liver organoid (DM-LO) and primary human hepatocyte (PHH), respectively (FIG. 16).

### Confirmation of Drug Metabolism Activity

As a result of analyzing the activity of major drug metabolizing enzymes using the P450-Glo Assay Kit (Promega), it was confirmed that the activity of CYP3A4 (i.e., a drug metabolizing enzyme) was increased 2.46-fold, and the activity of CYP1A2 was increased 3.77-fold compared to the differentiation medium-liver organoid (DM-LO) (FIG. 17).

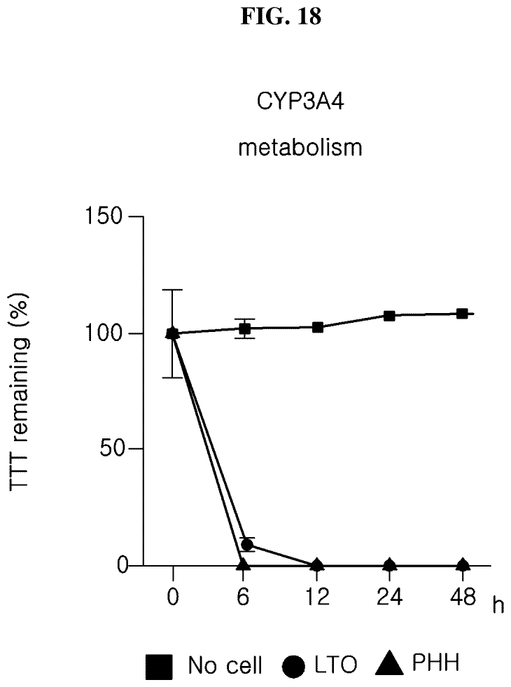

In particular, it was confirmed that testosterone metabolism occurred well at a level similar to that of primary human hepatocytes (PHHs), which are gold standard cells (FIG. 18).

### Confirmation of Glycogen Storage Ability

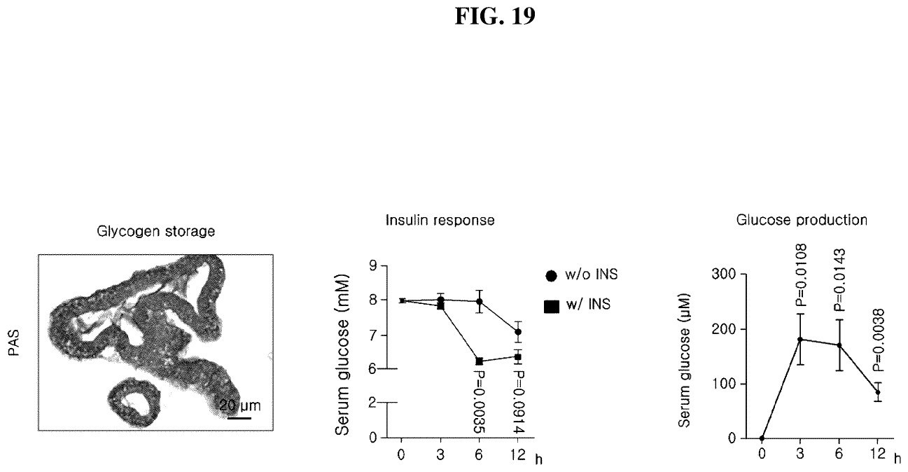

Glycogen storage ability was confirmed by PAS staining, and it was confirmed that glucose metabolic ability, such as insulin responsiveness and gluconeogenesis, was well maintained (FIG. 19).

### Confirmation of Lipid Metabolism

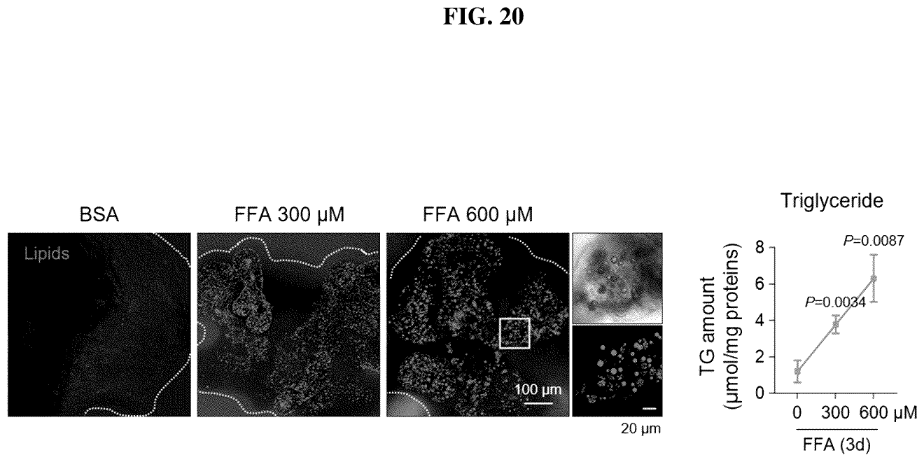

It was confirmed that after free fatty acid (FFA) treatment, lipid droplets stained with Nile red within the cells were increased, and the amount of triglycerides was significantly increased in a concentration-dependent manner, thereby also confirming that lipid metabolism (fat accumulation function) was well performed (FIG. 20).

### Confirmation of Functionality of Bile Ducts and Blood Vessels

In order to confirm whether the bile ducts and blood vessels formed in the liver tissue-like organoids (LTOs) have not only a structure but also an actual function, the organoids were observed after treating with CLF (i.e., a bile-like substance) and dextran (i.e., a fluorescent particle).

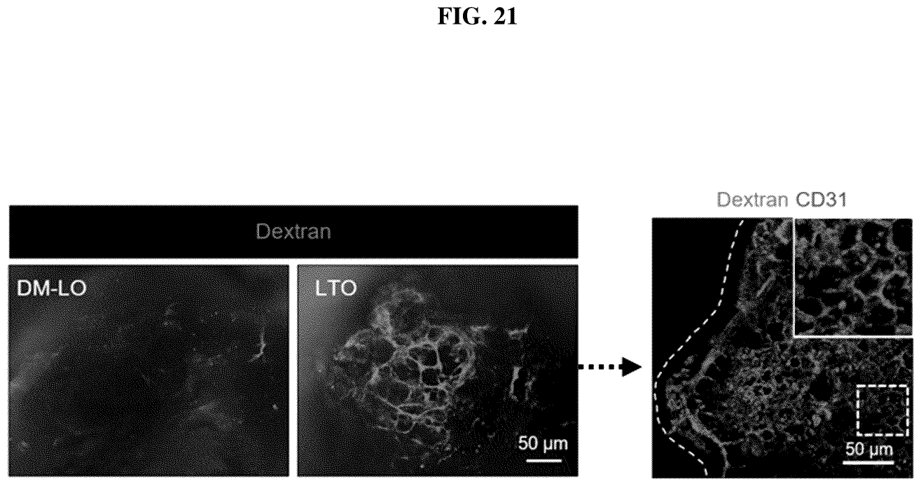

It was confirmed that since the differentiation medium-liver organoid (DM-LO) does not have a vascular structure, dextran was not perfused into the organoids, whereas in the liver tissue-like organoids (LTOs), dextran was well perfused into the liver along the CD31⁺ blood vessels (FIG. 21).

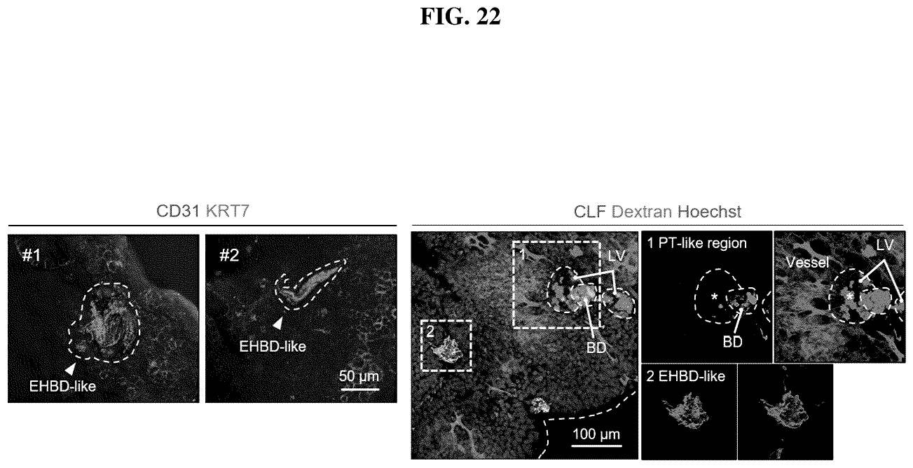

Additionally, it was confirmed that CLF was introduced into the bile ducts stained with KRT7, and dextran was perfused into the vascular structures stained with CD31, thereby confirming that the bile ducts and vascular structures formed in the liver tissue-like organoids (LTOs) were well maintaining the functionality of bile and substance movement like the liver tissue in the body (FIG. 22).

### Experimental Example 2. Evaluation of Drug Toxicity and Efficacy Using Liver Organoids

In the case of a model of the differentiation medium-liver organoid (DM-LO), the vascular structure was deficient, and their structural maturity was low even when non-parenchymal cells, including vascular endothelial cells, were present; therefore, there were limitations in analyzing the structural and functional interactions between parenchymal cells and non-parenchymal cells of the liver, or in accurately analyzing the mechanisms of liver disease and the toxicity and efficacy of drugs based on these interactions.

### Assessment of Toxicity of Busulfan Using Liver Tissue-Like Organoids (LTOs)

Busulfan is a drug used for treating chronic myeloid leukemia. Busulfan is known to have the side effect of damaging the blood vessels in the liver rather than the liver cells, but the exact mechanism for this side effect has not been identified, and to date, there has been no model similar to human liver tissue that can implement and confirm these responses.

Since the liver tissue-like organoid (LTO) model prepared in this Example has both parenchymal cells and non-parenchymal cells within the liver tissue, and mature vascular structures and immune cells are present therein, and the toxicity mechanism of Busulfan was analyzed using the liver tissue-like organoid (LTO) model.

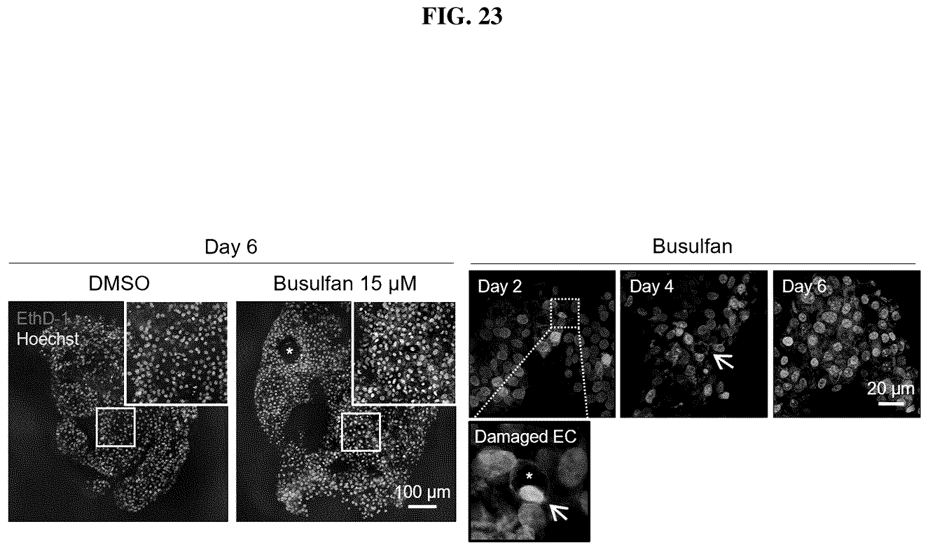

As a result of treating the liver tissue-like organoids (LTOs) with 15 μM of Busulfan every 24 hours for 6 days, it was confirmed that damaged vascular endothelial cells stained with EthD-1 were observed from Day 2 of Busulfan treatment, and then, damage was gradually induced from vascular cells to surrounding parenchymal cells, and from Day 6 of Busulfan treatment and thereafter, damage was induced throughout the organoids (FIG. 23).

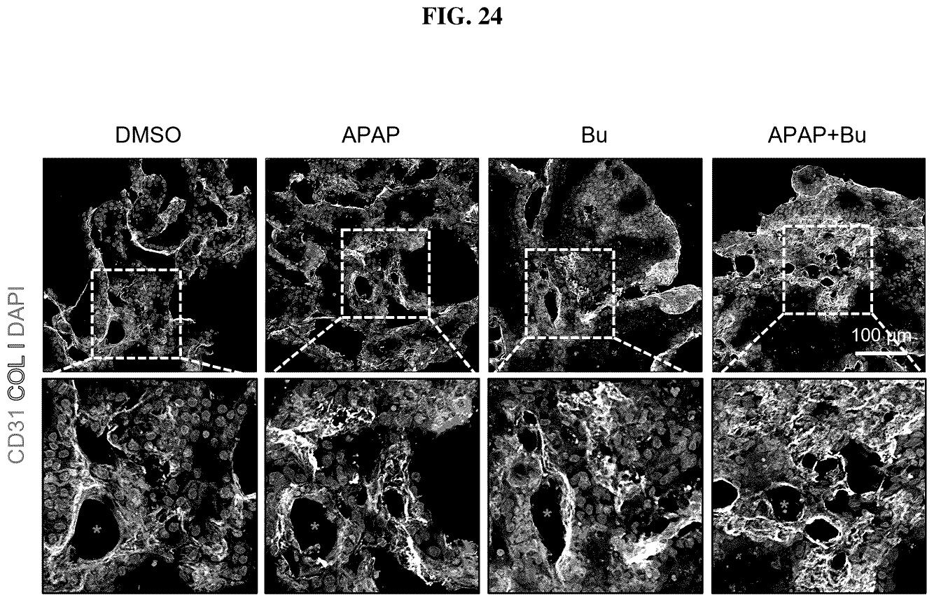

In addition, based on clinical results showing increased toxicity due to Busulfan when Busulfan and acetaminophen (APAP) were prescribed together, liver tissue-like organoids (LTOs) were treated with Busulfan and APAP together. Here, liver fibrosis due to liver damage progressed more rapidly than when treated with either Busulfan or APAP alone (FIG. 24).

### Evaluation of Efficacy of Resmetirom Using Liver Tissue-Like Organoids (LTOs)

In order to evaluate the efficacy of Resmetirom, which is a treatment for metabolic dysfunction-associated steatohepatitis (MASH), the assessment was performed using a disease model treated with free fatty acids (FFAs) in liver tissue-like organoids (LTOs).

As a result, Resmetirom was shown to reduce the accumulation of fat stained with

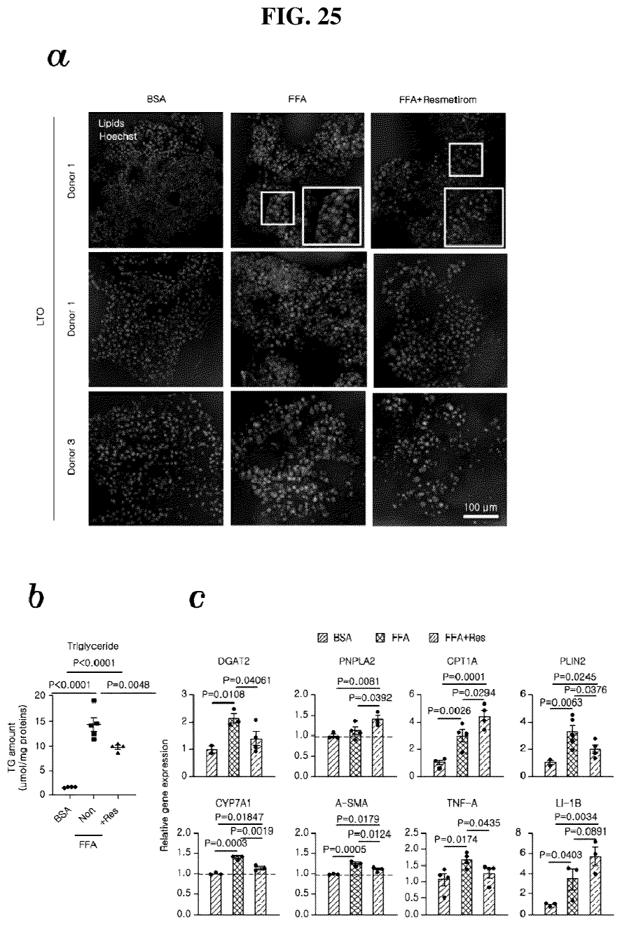

Nile red and the expression of fat synthesis-associated genes (DGAT2 and PLIN2), while increasing the expression of lipolytic activity genes (PNPLA2 and CPT1A) and reducing the amount of triglycerides (TG) (FIG. 25). These findings in liver tissue-like organoids (LTOs) are consistent with results observed in actual patients.

### Application of Liver Tissue-Like Organoids (LTOs)

As described above, the liver tissue-like organoids (LTOs) of the present invention are highly functional organoids that closely mimic liver morphogenesis and development. They contain various parenchymal and non-parenchymal cells present in liver tissue, as well as liver-specific bile ducts and vascular structures. As a result, they can be applied to modeling of liver diseases and evaluation of toxicity and efficacy of drugs with high accuracy.

1. A method for preparing a liver tissue-like organoid (LTO) from a stem cell-derived liver organoid,
comprising culturing the liver organoid in medium M1 comprising human bFGF, human VEGF-A, human PDGF-AB, human BMP4, human M-CSF and CHIR99021.

2. The method of claim 1, further comprising culturing the liver organoid in medium M2 comprising William's E Medium, P/S, Glutamax, HEPES, N2 Supplement, B27 Supplement with Vitamin A, Knockout SR, A83-01, DAPT, Dexamethasone, Y-27632 and human IL-3.

3. The method of claim 1, wherein in culturing in the medium M1, the medium M1 further comprises any one or more selected from the group consisting of:
Advanced DMEM/F12, William's E Medium, P/S, Glutamax, HEPES, N2 Supplement, B27 Supplement without Vitamin A, B27 Supplement with Vitamin A, ITS, Knockout SR, Nicotinamide, N-Acetylcysteine, Forskolin, A83-01, DAPT, Dexamethasone, Y-27632, [Leu]-gastrin I human, Oncostatin M, human HGF, human EGF, human BMP7, human FGF10, human FGF19 and human R-Spondin.

4. The method of claim 3, wherein the culturing in the medium M1 further comprises:
culturing in medium M1a, which further comprises human bFGF, human VEGF-A, human PDGF-AB, human BMP4, human M-CSF, CHIR99021, Advanced DMEM/F12, P/S, Glutamax, HEPES, N2 Supplement, B27 Supplement without Vitamin A, ITS, Nicotinamide, Forskolin, A83-01, [Leu]-gastrin I human, human HGF, human EGF, human BMP7, human FGF10 and human R-Spondin; and
culturing in medium M1b, which further comprises human bFGF, human VEGF-A, human PDGF-AB, human BMP4, human M-CSF, CHIR99021, Advanced DMEM/F12, P/S, Glutamax, HEPES, N2 Supplement, B27 Supplement with Vitamin A, A83-01, DAPT, Dexamethasone, [Leu]-gastrin I human, human HGF, human EGF, human BMP7 and human FGF19.

5. The method of claim 1, comprising:
(i) culturing the stem cell-derived liver organoid in suspension in a medium comprising extracellular matrix (ECM) from Day 0 (DO) to Day 4 (D4);
(ii) culturing the culture product obtained in step (i) in a state being embedded in the ECM from Day 4 (D4) to Day 12 (D12); and
(iii) culturing the culture product obtained in step (ii) under flow conditions, from Day 12 (D12) to Day 16 (D16).

6. The method of claim 1, wherein the stem cells are embryonic stem cells (ESCs) or induced pluripotent stem cells (iPSCs).

7. The method of claim 1, wherein the liver organoid is derived from stem cells by a culture method which comprises:
differentiating the stem cells into definitive endoderm (DE) cells; and
differentiating the definitive endoderm cells into hepatic endoderm (HE) cells.

8. A liver tissue-like organoid (LTO) prepared by the method of claim 1.

9. The liver tissue-like organoid (LTO) of claim 8, wherein the liver tissue-like organoid (LTO) expresses MRP2, BSEP, KRT7, KRT19, DESMIN, PDGFRB, CD31, LYVE1 and MARCO markers.

10. The liver tissue-like organoid (LTO) of claim 9, wherein the liver tissue-like organoid (LTO) further expresses one or more markers selected from the group consisting of HNF4A, ALB, ASS1, GS, PTPRC, SPN, CD11B, CD3, and CD56.

11. The liver tissue-like organoid (LTO) of claim 8, wherein in the liver tissue-like organoid (LTO), development of hepatocytes, cholangiocytes, hepatic stellate cells, endothelial cells and immune cells is observed.

12. The liver tissue-like organoid (LTO) of claim 9, wherein the liver tissue-like organoid (LTO) has a bile duct and a blood vessel structure, and in which bile can move through the bile duct and substance can move through the blood vessel.
